(19) United States
(12) Patent Application Publication
(10) Pub. No.: US 2009/0263884 A1
(43) Pub. Date: Oct. 22, 2009

(54) MULTI-ACTION DRAIN CLEANING COMPOSITION AND METHOD

(75) Inventor: RAJ MEHTA, WAYNE, PA (US)

Correspondence Address: DUANE MORRIS LLP - Philadelphia;IP DEPARTMENT, 30 SOUTH 17TH STREET, PHILADELPHIA, PA, 19103-4196, US

(73) Assignee: ORGANICA BIOTECH, INC., NORTH WALES, PA (US)

(21) Appl. No.: 12/107,578

(22) Filed: Apr. 22, 2008

(51) Int. Cl. C12N 1/20; C11D 7/06; C11D 10/00

(52) U.S. Cl. 435/252.1; 510/196; 435/264; 435/252.5

(57) ABSTRACT

The present invention relates to aspects of drain cleaning compositions, methods, and systems involving multiple levels of attack to facilitate decomposition of clogs in pipe systems. Aspects of the invention relate to a multi-action drain cleaning composition including chemical and biological components capable of producing an exothermic reaction, an oxidation reaction, and an enzymatic reaction. Generally, the chemical components act immediately to clear a clog in a pipe system and the biological components act over a longer period of time to provide longer-term maintenance of the pipe system. Additional aspects of the invention relate to a composition for use as a component of a multi-action drain cleaner that includes bacteria selectively cultured to subsist in a peroxygen-rich environment, a high-heat environment, and/or a high-pH environment. A bio-film for reducing or preventing a buildup of organic material in a pipe system including a matrix and a selectively cultured bacteria is also provided.

### BACKGROUND OF THE INVENTION

The invention relates to aspects of a drain cleaning compositions, methods, and systems involving multiple levels of attack to facilitate decomposition of clogs in pipe systems. More particularly, the invention relates to multi-action, chemical-biological drain cleaner compositions and methods of use of the same.

Drains and traps in the kitchen or bath can often be blocked or slowed by buildup of organic matter, such as grease, soap, hair, or food waste. Such organic blockage or partial blockage can be cleared mechanically or chemically with a variety of different trap and drain cleaners including concentrated acids or alkalis, oxidizing agents, and foaming or non-foaming combinations of these cleaners or biological systems. Examples of some of these are available from and identified in literature by Amway Corporation, Ada, Mich.; Ecolab Inc.; Enzyme Research & Development Corporation, Gilberts, Ill.; The Clorox Company, Oakland, Calif.; S.C. Johnson & Son, Inc., Racine, Wis.; Reckitt & Colman Inc., Montvale, N.J.; The Drackett Company, Cincinnati, Ohio; Acuity Brands, Inc., Atlanta, Ga.; Roebic Laboratories, Inc., Orange, Conn.; Hercules Chemical Company, Inc., Passaic, N.J.; and Jones-Stephens, Fla.

Drain cleaners have been formulated with a variety of active ingredients in an effort to remove the various materials that cause clogging or restriction of drains. Drain cleaners generally can be divided into four types, based on active components: (1) strong acids, such as sulfuric acids; (2) strong caustics, such as caustic soda, sodium hydroxide or caustic with aluminum chips; (3) strong oxidizing agents, such as sodium hypochlorite, which is catalyzed into action in an acid environment, and sodium percarbonate; and (4) enzymes, which degrade particular organic waste materials. None of these have proven to be fully satisfactory. Caustic and peroxygen technology has been taught by Bolan, U.S. Pat. No. 3,968,048, peroxide technology by Nakasone, U.S. Pat. No. 4,088,596, thickened hypochlorite technology by Dimond, U.S. Pat. No. 4,388,204, enzymes for hair dissolution, Jacobson, U.S. Pat. No. 4,540,506, caustic and hypochlorite by Taylor, U.S. Pat. No. 4,664,836, caustic and bacteria by Tobiason, U.S. Pat. No. 5,264,146, and halogen based oxidizing technology by Steer, U.S. Pat. No. 5,630,883.

Cleaners based on acids, caustic agents, or oxidizing agents can clear drains more quickly than enzyme-based cleaners and can be considered emergency drain cleaners. Emergency cleaners are usually cleaners that take as short as 30 minutes and as long as eight hours to clear the drain.

Acid and caustic emergency drain cleaners clear drains by chemically breaking down the materials obstructing the drain passage. Initially, the dissolution of acid and caustic elements in the trapped water generates a significant amount of heat. During the breakdown, more heat is generated, which further melts or degrades the obstruction. Both acidic and caustic formulations can be thickened by adding foaming agents or thickening agents or by incorporating active metals. The thickened formulation can more effectively bring the active agents in contact with the blockage. A greater level of heat is also released to help with the chemical degradation. These cleaners are traditionally used to clear biological or food matter in kitchen drains. However, the caustic or acid cleaners are considered extremely dangerous and in many cases require the use of a professional plumber.

Oxidizing drain cleaners also commonly rely on chemically induced elevated temperature and gas release to attack blockages. Previous oxidizing drain cleaners have been formulated to focus on hair and soap removal in bath drains. Oxidizing drain cleaners include those based on hypochlorite and percarbonate, but require the use of highly caustic or acidic environments to be effective. These cleaners have exhibited drawbacks such as incomplete removal of the blockage and/or slow action times. They can also be especially sensitive to atmospheric moisture.

Enzymatic drain cleaners typically utilize a combination of enzymes, bacterial cultures, surfactants, and fillers to clear blockage. This type of cleaner generally takes longer to react than caustic, acidic, or oxidizing cleaners and operates at much lower temperatures. They typically do not generate gas or cause foaming to occur. Without the increased temperature to melt or degrade the blockage or the gas release to penetrate the blockage, enzyme based cleaners generally clear drains at a much slower rate. The lack of immediate reaction puts these cleaners in the class of maintenance cleaners, which operate over a much longer time frame than emergency cleaners. Enzymatic drain cleaners traditionally have been incompatible with highly acidic, highly basic, and oxidizing formulations, which tend to render the enzymes or cultures ineffective.

While existing emergency drain cleaners can offer a quick solution to a clogged drain, depending on the nature and degree of obstruction, they have significant drawbacks. Their high degree of reactivity and potentially hazardous liquid nature can present safety concerns to the user. In addition, the extreme pH levels of these cleaners, higher than 12-13 on the base side and lower than 2-3 on the acid side, coupled with amount of heat and gas produced, can be potentially harmful and inconvenient to handle. Retail companies are continually restricting access of these materials to the consumer with a focus or restriction of sale to professional plumbers or contractors. Because most commercial emergency drain cleaners are liquid in nature, there is a danger of splashing or spilling or, in the extreme, accidental ingestion. The industry response to these dangers has been to produce milder, less reactive drain cleaners, which although somewhat safer, are still liquids and are often ineffective, leading the consumer to introduce other chemicals in an attempt to complete the drain cleaning process. Also, if the chemical cleaner is not effective, the user is left with a pipe filled with potentially dangerous materials. Therefore, some emergency cleaners are not generally available to the public, but only available to professional plumbers.

Enzyme-based cleaners can eliminate some of these inconveniences. However, their slower rate of reaction and decomposition place them in the maintenance cleaner category and, therefore, not suitable for situations requiring immediate reaction. Also, most biologic drain cleaners or those based on surfactants and other biologics that require some flow through the plug obstruction in order to be effective. Furthermore, enzyme-based cleaners are only useful when the obstruction is made of organic matters; therefore they have limited usefulness compared to other cleaners.

Further background information may be found in the following references: for bacterial or enzyme-based drain cleaners, the relevant prior art includes U.S. Pat. Nos. 6,638,902, 6,624,132, 5,464,766, and 5,225,083; for hypochlorite based drain cleaners, U.S. Pat. Nos. 6,638,900, 6,479,444, 5,630,883, 5,624,891, 5,931,172, 4,664,836, and 4,388,204; for active oxygen based drain cleaners, U.S. Pat. Nos. 6,413,927, 4,395,344, 4,206,068, 4,088,596, 4,060,494, 4,058,474, and 3,968,048; for acid drain cleaners, U.S. Pat. Nos. 5,721,203, 4,778,617, 4,666,625, and 4,453,983; and for caustic drain cleaners, U.S. Pat. Nos. 5,624,891, 7,246,628, 6,136,768, 5,624,891, 5,008,029, 4,587,032, 4,206,068, 4,080,305, 4,058,474, and 3,968,048. Other prior art has disclosed the concept of thermal stable bacteria as it applies to the chemical production of enzymes, preservation of food products, and the production of microorganism, but not in the area of drain cleaners: U.S. Pat. Nos. 3,272,717, 4,250,263, 4,332,895, 4,761,374, 5,059,430, 5,209,783, 5,545,548, and 5,945,325. The contents of these patents are incorporated herein by reference.

Accordingly, it is desirable to provide a multi-action drain cleaner that includes the short term emergency performance of, e.g., an active oxygen component, but that also includes a stable bacterial component to provide longer term maintenance protection, thereby overcoming the drawbacks of the prior art.

### SUMMARY

Generally speaking, in accordance with the invention, aspects of a drain cleaning composition, method, and system involving multiple levels of attack to facilitate decomposition of clogs are provided.

The present invention relates to a multi-action drain cleaning composition including a first component capable of producing an exothermic reaction, a second component capable of producing an oxidation reaction, and a third component capable of producing an enzymatic reaction. In some embodiments, the first component includes at least one chemical component, the second component includes at least one chemical component, and the third component includes at least one biological component. In these embodiments, the chemical components act immediately to clear a clog in a pipe system and the biological component acts over a longer period of time to provide longer-term maintenance of the pipe system.

The present invention relates to a composition for use as a component of a multi-action drain cleaner that includes at least one bacterium selectively cultured to subsist in a peroxygen-rich environment, a high-heat environment, and/or a high-pH environment.

The present invention further relates to a bio-film for reducing or preventing a buildup of organic material in a pipe system including a matrix and bacteria selectively cultured to subsist in a peroxygen-rich environment, a high-heat environment, and/or a high-pH environment.

The present invention further relates to a pipe system modified to reduce or prevent a buildup of organic material including a bio-film that includes a matrix and bacteria selectively cultured to subsist in a peroxygen-rich environment, a high-heat environment, and/or a high-pH environment.

The present invention further relates to a method for treating a clog in a pipe system and for reducing or preventing a buildup of organic material in the pipe system.

The present invention further relates to a method for selectively culturing at least one bacterium to subsist in a peroxygen-rich environment, a high-heat environment, and/or a high-pH environment.

The present invention further relates to a method for creating a bio-film for reducing or preventing a buildup of organic material in a pipe system.

The present invention further relates to a method for creating a pipe system with a bio-film for reducing or preventing a buildup of organic material in the pipe system.

Embodiments of the invention provide a number of advantages over prior art drain cleaners. For example, known conventional cleaners utilizing percarbonate technology include the presence of an acid, preferably a dry acid, to react and generate oxygen from an alkali percarbonate or perborate. This generates heat and gas to attack the clog. On the other hand, compositions in accordance with preferred embodiments of the invention need only the presence of a heat source, a carbohydrate source, and water. They can be offered at more moderate pH levels.

Unlike traditional caustic or acidic cleaners, preferred embodiments of the invention do not depend on a high concentration of strong acid, such as sulfuric acid or strong base, such as sodium hydroxide, to dislodge the plug. Instead, the percentage of caustic and aluminum combination can be less than ten percent (10%) by weight of the total composition. The benefits of a relatively lower concentration of caustic include enhanced safety in handling, improved convenience in storage, delivery, and application, and a generally more environmentally-friendly product.

### BRIEF DESCRIPTION OF THE DRAWINGS

For a fuller understanding of the invention, reference is had to the following description taken in connection with the accompanying drawing, in which:

FIG. 1 is a graph showing the cumulative result of 60 P-trap plug tests, performed over an extended period of time. The x-axis represents the time the cleaner took to remove the test plug and the y-axis represents number of tests conducted.

FIG. 2 is a cross-sectional view of a pipe system with a bio-film for reducing or preventing a buildup of organic material in the pipe system, in accordance with certain aspects of the invention.

FIG. 3 is a perspective view of a pipe system with a bio-film for reducing or preventing a buildup of organic material in the pipe system with a portion of the bio-film cut away for clarity, in accordance with certain aspects of the invention.

The above-identified drawings are not necessarily drawn to scale.

### DESCRIPTION

The present invention concerns aspects of a multi-action drain cleaning composition, method, and system involving multiple levels of attack to facilitate decomposition of clogs.

One embodiment of the claimed invention is a drain cleaner composition with chemical and biological components that, in combination, provide three levels of attack on a clog or plug within a drain, piping, tubing, or plumbing system. Upon introduction to a clogged drain, a first component of the composition produces an exothermic reaction from at least one chemical component, a second component of the composition produces an oxidation reaction from at least one chemical component, and the third component of the composition produces an enzymatic reaction from at least one biological component.

The first component of the drain cleaner composition, which produces an exothermic reaction from a chemical component such as a chemical heat source, preferably includes the reaction of at least one strong base and at least one metal, such as caustic (sodium hydroxide) and aluminum chips. In comparison to traditional caustic and active metal drain cleaners, where the active agent is usually in excess of eighty percent (80%) by weight, in a preferred embodiment, only small quantities of the exothermic chemical heat source are needed, e.g., less than about eight percent (8%). This component can comprise less than about ten percent (10%), and preferably less than about eight percent (8%), of the composition. This chemical heat-generating component is advantageously formulated to raise the temperature at the clog to above about 50° C. Preferably, this initial heat generation raises the water temperature to about 60° C. or higher.

The second component, which produces an oxidation reaction from a chemical component or source, preferably includes at least one peroxygen compound, which dissolves in the water above the plug and reacts with at least one organic substrate in the plug or added to the composition as a filler, such as a carbohydrate, to convert the peroxygen component to an organic peracid. The reaction of the peroxygen compound with either a carbohydrate additive or the organic matter within the plug provides an additional boost in temperature plus the generation of gas to fracture the plug. This component may, thus, react more swiftly with an organic plug blockage than the first component and also may bring the entire formulation to a level of higher heat, typically about 80° C. or higher, in a shorter period of time than with the first component alone. The heat generated by the first component heat source and the second component peroxygen compound initiates the oxidation of any carbohydrate or degradation of the organic material at the clog site by the peroxygen compound, thereby initializing a second level of attack on the clog. Oxygen and more heat are generated. This can raise the water temperature at the clog to about 80-90° C. The oxygen gas can act to fracture and channel through the blockage. The peroxygen compound continues to react with and decompose the material at the clog.

The third component, which produces an enzymatic reaction from a biological component, provides a third level of attack by at least one bacterium and enzymes. While most bacteria cannot survive in a peroxygen-rich, high-heat, and high-pH environment such as that effected by the first and second components of the composition, the bacteria used in preferred embodiments are specifically cultivated to subsist in such an environment. The foaming of the drain cleaning composition above the plug causes water insoluble bacteria to float to the surface of the water. Once the plug has been cleared by the first and second components, the water above the plug is then free to pass through the pipe and drain, thereby coating the walls with bacteria, which in turn produces a bio-film that keeps the drain free from further clogs. Test plates demonstrate that greater than about ninety percent (90%) of the engineered bacterial composition survives exposure to the heat, caustic, and peroxygen chemicals in the drain. Some embodiments may include grease-attacking bacteria. The third component may also include a carbohydrate.

The combination of the first and second chemical components with the third biological component achieves the advantage of combining thermal and oxidizing power with biological power, resulting in a safe, solid cleaner composition suitable for both bath and kitchen drains.

Embodiments of the drain cleaner composition may also include additives, such as organic and inorganic diluents like dextrose, bran, sodium carbonate, and perlite. Additionally, various types of builders, diluents, surfactants, drying agents, fatty acids, and fatty acid esters can be added to further improve the efficacy and safety of the composition. These additives can be selected based on reactivity with the peroxygen compound to generate heat and oxygen. Surfactants or fatty acid esters can also be included in the composition to facilitate the removal of the plug as slip agents.

In preferred embodiments, the drain cleaner composition is delivered as a safe and easy-to-handle dry blend. The composition may be delivered as granules, powder, capsules, tablets, or cakes. For the purpose of an emergency drain cleaner, the composition is preferably in a granular form to maximize its reactivity and effectiveness. In tablet form, the composition most advantageously can be used as a drain or pipe cleaner, but can also be used as a garbage disposal cleaner, toilet bowl or urinal cleaner or other applications involving a system where a still water drain may occur. A dry blend of a composition in accordance with preferred embodiments of the invention does not require special packaging, can be stored in a plastic or glass bottle with a child-proof or -resistant cap, and can be formulated to be moisture stable.

As a dry blend or otherwise, the composition may be applied to a variety of drain, piping, tubing, and plumbing systems and their components, including garbage disposals, washing machines, ice machines, and other areas where drain lines can become clogged.

Preferred embodiments of the invention can be formulated so that most of the dry blend cleaner composed of additives and active agents readily sink to the bottom of the trap when added to a slowed or stopped drain which contains still water. This replaces the typical thickened agents employed in liquid emergency drain cleaners. In contrast, the longer acting component—the bacteria—rise to the surface of the still water. After a brief period of incubation, the strong base and metal combination reacts with standing water to produce heat, initiating the peroxygen compound's reaction with any carbohydrate components of the clog or with other components of the organic blockage to generate moderate heat, gas, and foam. The heat, gas, and foam generated are sufficient to react with and dislodge the organic matter. The removal of the plug is facilitated by the presence of the surfactants and slip agents. The peroxygen-stable bacteria form a bio-film that is substantially compatible with the environment effected by the first and second component and that provides a stable, long-lasting environment within the pipe to reduce or prevent further buildup of organic waste matter. The combination is fast, safe and effective in that the organic blockage may be dislodged within about 15-30 minutes of application.

A drain cleaner composition in accordance with the preferred embodiments of the invention can generate greater than about 340 cal/gram of heat, but less than about 811 cal/gram of heat. Preferably, about 600-700 cal/gram of heat is generated. The maximum temperature achieved by the drain cleaner reaction should be greater than about 30° C. but less than about 86° C., preferably between about 77° C. and 86° C., and most preferably about 80° C. The maximum temperature should be sustained for more than one minute, preferably at least about 90 seconds.

In addition, 56 grams of a drain cleaner composition in accordance with the preferred embodiments of the invention can generate a foam height greater than about 100 mL, preferably about 400 mL. With these concentrations of active agents and the temperatures reached and maintained, the composition can dislodge and dissolve hair. Traditional hair clog removers generally require heavy concentration of caustic and hypochlorite. Without hypochlorites, the composition can be delivered as a solid, thus increasing its safety in comparison to liquid cleaners containing caustic and hypochlorite.

A drain cleaner composition in accordance with the invention is made by a dry blending process using a Paterson-Kelley mixer or tumbler mixer. The preferred method is using a Paterson-Kelley mixer. Because of the sensitivity of the peroxygen compound to water or moisture, it is preferable to avoid high humidity in the process of preparation. Conventional coating agents can also be employed to enhance the safety of the composition. However, because preferred embodiments of the composition can be dry blended in air to be moisture stable, they do not require special packaging. Once the dry blend is prepared, the final formulation can be stored in standard containers with child-proof caps. The bottle can be opened and closed to utilize appropriate amounts of the composition per treatment without concern that moisture from the air will degrade the performance of the product. In contrast, conventional blends that contain dry acids and percarbonates/carbonates typically require special packaging due to the extreme moisture sensitivity.

In an embodiment of the composition, the first component, i.e., chemical heat source, comprises about zero to thirty percent (0-30%), preferably about five-tenths of a percent to ten percent (0.5-10%), by weight caustic and about five-tenths of a percent to ten percent (0.5-10%) by weight aluminum. The preferred ratio by weight of caustic to aluminum is about 50:50. Ratios of about 45:55 to 55:45 caustic:aluminum are also effective. The caustic can either be flake or bead form, preferably in bead form. The aluminum can be about ninety-seven percent (97%) by weight pure aluminum and can come in a variety of chip shapes. The preferred shape is rectangular, approximately 1 mm×3 mm in size. The aluminum flakes or chips offered by Transmet Corporation, Columbus, Ohio, product K-101, is a suitable material for use.

The second component, i.e., the peroxygen compound, is about five to eighty percent (5-80%) by weight, preferably about ten to seventy percent (10-70%) by weight, and most preferably about fifteen to thirty percent (15-30%) by weight of the drain cleaner composition. Preferred peroxygen compounds include salts, such as alkaline metal and alkaline earth metal salts, of percarbonates, persulfates, and perborates, and mixtures thereof, such as sodium percarbonate, sodium persulfate, sodium perborate, and mixtures thereof. Preferred compounds include peroxygen analogs of simple alkaline or alkaline earth metal salts of percarbonates that are utilized in traditional drain cleaners. The most preferred peroxygen compound is sodium percarbonate, having the formula 2Na₂CO₃.3H₂O₂. Sodium percarbonate is available from Burlington Chemical Co., Burlington, N.J.

The third component, i.e., the biologic component, is about five-hundredths of a percent to twenty-five percent (0.05-25%) by weight and preferably about one-tenth of a percent to ten percent (0.1-10%) by weight of the drain cleaner. Biologic components in accordance with embodiments of the invention comprise an organic diluent, such as a carbohydrate, and at least one bacterium developed to be stable in the presence of a peroxygen compound and caustics. The preferred powdered enzyme/bacterial fermentation product can include enzymes, preservatives, activators, aerobic, and non-aerobic bacteria and buffers. The diluent is typically about eighty to ninety-eight percent (80-98%) of the bacterial additive.

Suitable bacteria for use in the biologic component of the drain cleaner composition include those which express anti-clog material, such as several strains of spore-forming Bacillus sp. and high-oxygen-tolerant mutants developed therefrom. Desirable enzymes produced by the bacteria include lipases, amylases, cellulases, and proteases. Lignin-degrading bacteria are particularly useful.

High-oxygen-tolerant mutants of Bacillus sp. are cultivated using the gradient plate method with sodium percarbonate and dextrose or sodium carbonate. The gradient plate method is a classic method used to select and cultivate bacterial strains with desired characteristics. Strains are cultured in the desired environment, in this case, in the caustic and high peroxygen environment. Mutant cultures that are able to survive the harsh conditions and still express the desired enzymes are repeatedly cultivated in a medium with increasingly higher concentrations of the peroxygen compound and caustic (e.g., sodium hydroxide) until the resulting mutant is able to survive in the desired level of caustic and oxygen.

Examples of Bacillus strains used in the gradient plate method include selectively mutated versions of Bacillus subtilis ATCC 6051, ATCC 14415, and ATCC 35946; Bacillus lichenifonnis ATCC 6598 and ATCC 1194; and Bacillus polymyxa ATCC 12060. In addition to the examples noted here, other selectively mutated versions of Bacillus sp. or other oxygen-tolerant microbes that express suitable proteins can be used in this method without restriction. The advantage of utilizing oxygen-tolerant microbes is extended shelf life. Generally, spore formers have the longest rate of survival in such formulations.

Suitable enzymes of the class lipase, amylase, cellulase, and protease may also be incorporated into the drain cleaner composition. An example of the system includes a bacteria culture comprising about 0.1-15% Bacillus, about 0.1-15% protease, about 0.1-15% amylase, about 0.1-15% cellulase, and about 0.1-15% lipase. The bacteria can be blended in diluents such as bran, dextrose, sodium bicarbonate, or other similar media. The addition of an enzymatic system increases the efficacy of the drain cleaner by coating the sides of the pipes and inhibiting the clogs from reforming, therefore acting as a long-term cleaning agent.

In accordance with the foregoing, suitable bacteria have the characteristic that once employed in the drain cleaning action, the bacteria will adhere to the internal surfaces of pipes in a pipe system, such as, for example, a drain system. Bacterial samples taken from various segments of pipes to which the drain cleaning composition is applied indicate more than 90% of the bacteria survive the initial drain treatment process and are thus able to support the longer term maintenance process. After the initial drain treatment process, the bacteria adhere to the internal surfaces of the pipes of the pipe system. The bacteria begin to germinate and produce self-sustaining colonies within a matrix that includes substances excreted by the bacteria. The excreted substances may include polymeric compounds such as polysaccharides. The bacteria and matrix constitute a bio-film on the interior surface of the pipes of the pipe system. The bio-film may be characterized by surface attachment, structural heterogeneity, genetic diversity, and complex microorganism interaction. Within the bio-film, the matrix protects the bacteria and other microorganisms that may become integrated into the bio-film. The bio-film is substantially compatible with the environment effected by the first and second components and the bio-film is maintained by the nutrient-rich aqueous environment produced by the water and organic material moving through the pipes with typical use. The bacteria produce enzymes to metabolize as substrate the organic material that would otherwise deposit on the surface of the pipes, accumulate, and cause a clog. By this process, the bio-film provides a long-term maintenance solution preventing the buildup of organic and other material that would otherwise clog the pipes of the pipe system. A modified pipe system comprising the bio-film is thus realized. The bacteria become acclimated to the environment of the pipe system and the colonies continue to grow, spreading the bio-film throughout the pipe system. Subsequent emergency drain treatments do not adversely affect the bio-film because the bacteria are designed to be tolerant of the extreme conditions produced by, e.g., a chemical heat source and peroxygen compound such as, e.g., extreme pH, high heat, and high oxygen. Waste produced by the enzymatic metabolization of the organic substrate that does not subsist in the matrix is flushed away with water moving through the pipe system with typical use or is treated with suitable cleaning agents or solvents. Excessive expansion or buildup of the bio-film or the matrix of the bio-film is also advantageously controlled by the use of suitable cleaning agents or solvents. The bio-film may be sustained or fortified by the periodic supply of suitable agents to the plumbing system. For example, a composition comprising nutrients, oxygen-producing agents, or heat-producing agents, or any other agent that may effect a condition favorable to the bacteria, may be added to the pipe system to maintain the bio-film over long periods of time when normal use of the pipes would not otherwise adequately sustain the bio-film.

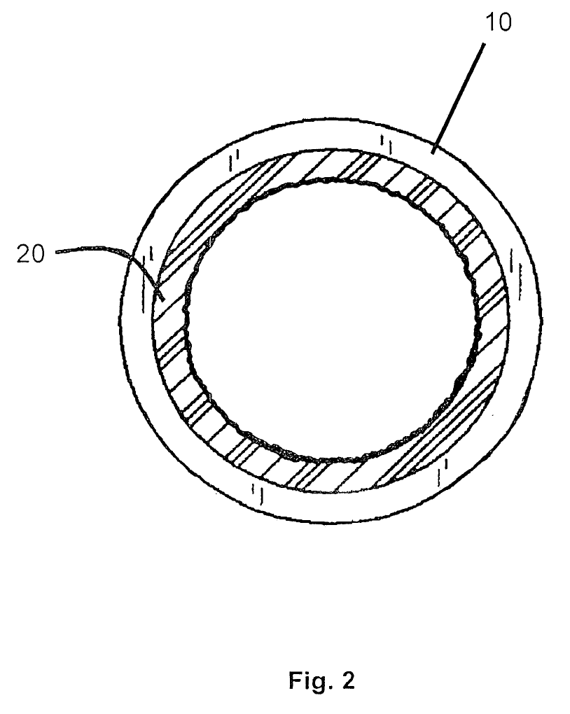

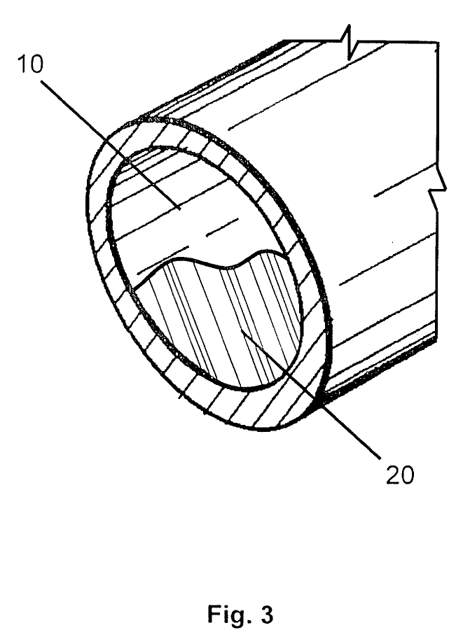

Referring to FIGS. 2 and 3, bio-film 20 is disposed along the interior surface of pipe 10 of a pipe system.

As described above, certain embodiments of the drain cleaner composition may include additives to improve the efficacy and safety of the composition.

One such additive is a carbohydrate source, e.g., a simple sugar, such as dextrose. Fructose, glucose, sucrose, and other sugars are also acceptable. Other acceptable carbohydrates include bran, wood flour, flaxseed, and lignin, and combinations thereof. The carbohydrate may be about five-tenths of a percent to twenty-five percent (0.5-25%) by weight of the composition and is preferably about one-tenth of a percent to fifteen percent (0.1-15%) by weight of the composition. The carbohydrate improves the reactivity of the peroxygen compound. The peroxygen compound reacts with the carbohydrate, creating an exothermic reaction, releasing heat and oxygen. This causes the peroxygen compound to react with the organic blockage, generating more heat and oxygen, which further stimulates the reaction. The oxygen expands and penetrates the blockage to facilitate removal, creating more surface area and further enhancing the reaction. The carbohydrate can also serve as an organic diluent to support and dilute the bacteria and/or enzyme.

Other additives include a surfactant system or a fatty acid ester, which can be added to the drain cleaner composition to enhance the removal of the plug by acting as a slip agent. Suitable surfactants include the sodium lauryl sulfate family, and the fatty acid ester can be taken from any commercial class of mono-, di-, tri-alcohols, or soybean derivatives. The surfactant system may represent about five-hundredth of a percent to ten percent (0.05-10%) by weight, preferably about one-tenth of a percent to five percent (0.1-5%) by weight, and most preferably about three-tenths of a percent (0.3%) by weight of the drain cleaner composition.

Suitable surfactant systems include both foaming surfactants and emulsifying surfactants to facilitate the penetration and degradation of the blockage. Foaming surfactants can include a number of different classes, such as anionic surfactants, amphoteric surfactants, and nonionic surfactants, as well as combinations of these surfactants. A preferred surfactant system utilizes anionic surfactants. Examples of surfactants include: (1) alkali metal salts of ethoxylated alcohols, such as those having the general formula RO(CH₂CH₂O)ₙSO₃⁻M⁺, where R represents an aliphatic group preferably of 8-18 carbon atoms, M+represents an alkali metal, preferably sodium, potassium, or lithium, and n is about 1-6; (2) alkali metal salts of an N-alkyl, N-fatty acyl amino acid, such as those having the general formula R₁—C(O)—N(R₂)—CH₂—COO⁻M⁺, where R₁ is a linear or branched chain of preferably 1 to 6 carbon atoms and R₂ preferably has 6-18 carbon atoms; (3) alkali metal salts of alkyl sulfates, such as those having the general formula R₃SO₄⁻M⁺, where R₃ preferably does not include aromatic groups; and (4) neutral surfactants, such as those having the general formula (CH₂CH₂O)ₙ. Suitable emulsifying surfactants include dry sodium lauryl sulfate, which can be obtained from Stepan Chemical Company of Northfield, Ill., under the trade name Stepanol ME-Dry. The fatty acid esters of simple alcohols, propylene glycol, or glycerine can be employed where the fatty acid is any of a number of monocarboxylic acids having chain lengths varying from C₁₀-C₂₄.

Other additives to the drain cleaner composition include inorganic diluents. The inorganic diluents may be about five percent to sixty percent (5-60%) by weight of the composition and are most preferably about ten percent to fifty percent (10-50%). Suitable inorganic diluents include sodium carbonate, sodium bicarbonate, sodium aluminum silicate, and perlite, and combinations thereof. Phosphates of sodium and potassium can also be included, but should be water soluble. For example, sodium tripolyphosphate is a preferred inorganic diluent. Sodium tripolyphosphate can increase the alkalinity of the drain cleaner composition and can help remove or emulsify the soap, grease, or fat. The inorganic diluents do not interfere with other chemical reactions, but are added to modify or buffer the pH balance. Sodium carbonate easily dissolves in water. Although perlite is insoluble in water, because of its density it floats on the top of the still water separated from the active ingredients in the drain, allowing the active ingredients to react with the pipe blockage. A preferred form of perlite is Perlite 27S, supplied by American Perlite, North Hollywood, Calif. An example of acceptable sodium tripolyphosphate is a standard industrial grade offered by FMC Corporation of Philadelphia, Pa., for use in detergents.

Other additives, such as colorants, foam suppressants, or fragrances, can also be added to enhance the appearance or presentation of the drain cleaner composition. If the cleaner is in the form of a tablet, anti-caking agents, binding agents, abrasive agents, and corrosion inhibitors can also be used. These all assist in the removal of foreign matter from either the drain, pipe, or garbage disposal. The cleaner can also be in powder form. Furthermore, the cleaner can also include a defoamer such as Rhoadline DD770, available from Rhodia of Cranbury, N.J.

### EXAMPLES

The following examples are provided in order to demonstrate the effectiveness of preferred embodiments and how they compare to commercially available drain cleaners on the market today. The examples are presented for purposes of illustration only and are not intended to be construed as limiting.

### Example 1

### Preparation of Cleaners

A sample of a cleaner in accordance with an embodiment of the invention (“Cleaner 1”), a sample of a cleaner in accordance with an embodiment of the invention without the biologic component and carbohydrate additive (“Cleaner 2”), and five other commercially available cleaners were tested and compared, using various test procedures previously described in patent literature. The compositions of all the cleaners are detailed below. Cleaner 1 and Cleaner 2 were prepared by premixing the surfactant, silicate, caustic, and aluminum chips in a Paterson-Kelley mixer in a dry environment and then incorporating this blend into a final mix with the sodium percarbonate, sodium carbonate, and, for Cleaner 1, bacteria and dextrose.

Cleaner 1 was prepared with components within the following general ranges:

| Component | CAS# | % by Wgt | Supplier |
|---|---|---|---|
| Sodium percarbonate | 5968-11-6 | 10-60 | Burlington Chemical |
| Sodium carbonate | 5968-11-6 | 30-80 | FMC Corp. |
| Sodium lauryl sulfate | 151-21-3 | 0.1-3.0 | Stepan Chemical |
| Lignin-degrading | ?? | 1-10 | Organica Corp. |
| bacteria/dextrose | | | |
| Caustic beads | 1310-73-2 | 5.0-15.0 | N/A |
| Aluminum granules | 7429-90-5 | 1.0-5.0 | Transmet Corp. |
| Sodium aluminum | 73987-94-7 | 0.1-2.0 | PC Corp. |
| silicate | | | |

As explained above, the dry surfactant can be complemented with or replaced by a dry fatty acid ester typified by saturated or unsaturated fatty acid esters of propylene glycol or glycerin.

Specifically, Cleaner 1 was prepared as follows:

| Component | CAS# | % by Wgt | Supplier |
|---|---|---|---|
| Sodium percarbonate | 5968-11-6 | 25 | Burlington Chemical |
| Sodium carbonate | 5968-11-6 | 60 | FMC Corp. |
| Sodium lauryl sulfate | 151-21-3 | 0.2 | Stepan Chemical |
| Lignin-degrading | ?? | 3 | Organica Corp. |
| bacteria/dextrose | | | |
| Caustic beads | 1310-73-2 | 8.3 | N/A |
| Aluminum granules | 7429-90-5 | 3.0 | Transmet Corp. |
| Sodium aluminum | 73987-94-7 | 0.5 | PC Corp. |
| silicate | | | |

Cleaner 2 had the same composition as Cleaner 1, but did not include the lignin-degrading bacteria biologic component and dextrose additive. The weight of bacteria and dextrose was replaced by sodium carbonate.

Cleaner 3 was a sulfuric acid based drain cleaner marketed under the designation Flow Easy™ manufactured by Jones-Stephens of Florida.

Cleaner 4 was a caustic/aluminum drain cleaner marketed under the designation CloroClean™ by Hercules Chemical Company, Inc. of Passaic, N.J.

Cleaner 5 was a liquid caustic drain cleaner marketed under the designation Mr. Plumber™ by SC Johnson of Racine, Wis.

Cleaner 6 was a caustic potassium hydroxide and sodium hypochlorite drain cleaner marketed under the designation 10 Min Hair Remover™ by SC Johnson of Racine, Wis.

Cleaner 7 was a peroxygen based drain cleaner marketed under the designation Foamy Mr. Plumber™ by SC Johnson of Racine, Wis.

Cleaner 8 was a cleaner with the same ingredients as Cleaner 1, but without caustic and aluminum.

Cleaner 9 was a cleaner with the same ingredients as Cleaner 1, but including an additional 10% by weight sodium tripolyphosphate (STPP).

### Example 2

### Heat Generation

One important factor for evaluating drainer cleaner performance is heat generation, as described in U.S. Pat. No. 4,206,068, incorporated herein by reference. To study heat generation, a recommended amount of each cleaner for 100 mg of water was added to the water in a calorimeter to accurately measure the heat generated. The temperature change, time to reach maximum temperature, and duration of time which maximum temperature was sustained were measured and recorded in Table 1.

The temperature was monitored during the first 5 minutes of mixing. As seen from Table 1, Cleaner 2, which did not contain the biologic component and carbohydrate additive experienced no temperature increase due to lack of dextrose that acts to accelerate the reaction by serving as a reducing agent for the percarbonate oxidizing agent. The initial increase in temperature of the cleaning system when placed in water is due to the reaction of caustic/aluminum with water. If the caustic/aluminum is eliminated, the change in temperature profile is dramatic. For example, the test of Cleaner 8, which included the ingredients of Cleaner 1 except for the caustic/aluminum mixture, showed that the temperature increased at a slower rate and did not reach the optimum temperature.

As can be seen from Table 1 below, Cleaner 1 and Cleaner 9 provided superior heat for a longer period of time than competitive products tested. In particular, the resulting heat generated, measured in calories per gram, clearly demonstrated these compositions' excellent ability to reach and maintain a temperature necessary to degrade and fracture the plug, which as U.S. Pat. No. 4,206,068 teaches, is set at a desirable minimum of 250 cal/gm. Only Cleaner 1 and Cleaner 9 maintained a maximum temperature for 10 minutes before beginning to cool, thereby prolonging the window during which the active components of the composition react and degrade the blockage.

TABLE 1
HEAT GENERATION

| | | | | Time | |
|---|---|---|---|---|---|
| | | | Max | at | Heat |
| | | Time | Temp. | Max | Generated |
| Cleaner | Chemical Type | (s) | (° C.) | Temp. | (cal/gm) |
| Cleaner 1 | caustic/aluminum, | 360 | 90° C. | 10 min | 690 |
| | peroxygen, | | | | |
| | bacteria/carbohydrate | | | | |
| Cleaner 2 | caustic/aluminum, | 360 | 30° C. | N/A | <250 |
| | peroxygen | | | | |
| Cleaner 3 | sulfuric acid | 20 | 86° C. | 10 sec | 340 |
| Cleaner 4 | caustic/aluminum | 87 | 77° C. | 8 min | 811 |
| Cleaner 5 | caustic solution | 360 | 35° C. | N/A | <250 |
| Cleaner 6 | caustic and | 360 | 35° C. | N/A | <250 |
| | hypochlorite | | | | |
| Cleaner 7 | peroxygen | 360 | 35° C. | N/A | <250 |
| Cleaner 8 | Cleaner 1 without | 360 | 30° C. | N/A | <250 |
| | caustic/aluminum | | | | |
| Cleaner 9 | Cleaner 1 plus 10% | 360 | 90° C. | 10 min | 690 |
| | by weight STPP | | | | |

### Example 3

### Gas Generation and Foam Height

Gas generation is an important feature in the efficacy of a drain cleaner because gas can fracture a blockage. By sealing the top opening of the drain, the gas generated and the pressure build-up allows an accelerated elimination of the blockage. Unlike most acid and highly caustic drain cleaners, a drain cleaner composition in accordance with an embodiment of the invention produces gas.

Foam height is another important measurement in evaluating the effectiveness of a drain cleaner. Foam and surfactants help to fracture and dislodge the plug and promote more complete plug removal. While the foam rises along the drainpipe, active chemicals are deposited throughout the dimensional cross section of vertical and horizontal pipes. The foam also allows the active chemicals to move up the pipe to the drain area, where food particles, hair, and soap can lodge around the trap system. The foaming action also provides fissures in the drain blockage, through which the active drain cleaner can then penetrate more easily into the sides of the clog.

To measure the amount of gas generated, a 500 mL Erlenmeyer filter flask was stopped with a tube running from a discharge nipple to an inverted 1000 mL graduated cylinder containing water. After a test sample of cleaner was added to the flask along with 50 mL of water at 35° C., the mixture was shaken to disperse the solid particles and then kept at rest in a water bath. Gas generation was measured by the displacement of the water in the inverted graduated cylinder. The test was repeated for all the cleaners listed above.

To measure the amount of foam generated, foam was generated and measured in a 1000 mL graduated cylinder. A weighed quantity of the drain cleaner was placed in the bottom of the cylinder and 100 mL of water at 35° C. was added. After first measuring the height of the cleaner-water mixture in the cylinder, the mixture was stirred and then allowed to rest. The amount of time for foaming to occur and the height of the foam produced were measured.

Table 2 summarizes the result from the foam and gas experiments. Cleaner 1 produced a moderate amount of foam in a brief period of time, achieving a foam height of 400 mL. The foam persisted for 120 seconds. Without the presence of the bacteria and dextrose, as seen in the result for Cleaner 2, the gas evolution dropped significantly.

TABLE 2
GAS GENERATION/FOAM HEIGHT

| | | | | Time |
|---|---|---|---|---|
| | | Time to Produce | Foam | to Foam |
| Cleaner | Chemical Type | 0.4 L of Gas | Height | Height |
| Cleaner 1 | caustic/aluminum, | 58 seconds | 400 mL | 1 min |
| | peroxygen, | | | |
| | bacteria/ | | | |
| | carbohydrate | | | |
| Cleaner 2 | caustic/aluminum, | No gas evolution | None | N/A |
| | peroxygen | | | |
| Cleaner 3 | sulfuric acid | No gas evolution | None | N/A |
| Cleaner 4 | caustic/aluminum | No gas evolution | None | N/A |
| Cleaner 5 | caustic solution | No gas evolution | None | N/A |
| Cleaner 6 | caustic and | Very Low | None | N/A |
| | hypochlorite | | | |
| Cleaner 7 | peroxygen | Very Low | None | N/A |
| Cleaner 8 | Cleaner 1 without | Very low | 100 mL | >5 min |
| | caustic/aluminum | | | |
| Cleaner 9 | Cleaner 1 plus 10% | 60 seconds | 450 mL | 1 min |
| | by weight STPP | | | |

### Example 4

### Grease Dissolution

A number of grease dissolution protocols are mentioned in the prior art. Standard grease plugs are described in, e.g., U.S. Pat. Nos. 4,060,494 and 4,058,474, which are incorporated herein by reference. Three compositions of grease plugs listed in Table 3 below were used to test the cleaners. The compositions were generally similar to that provided in the prior art, however, modifications were made to focus on the use of common kitchen or bathroom ingredients.

TABLE 3
GREASE PLUG COMPOSITIONS

| Plug | | | |
|---|---|---|---|
| Composition 1 | Wgt (g) | Plug Composition 2 | Wgt (g) |
| Corn Starch | 10 | Lava soap | 2 |
| Gelatin | 12.5 | Gelatin | 12.5 |
| Agar | 3 | Agar | 3 |
| Bird Suet | 8 | Suet | 8 |
| Citric acid | 1 | Calcium Carbonate | 3 |
| Coffee grinds | 1 | Citric acid | 1 |
| Water | 550 | Water | 551 |

Plug Compositions 1 and 2 were prepared by first mixing the gelatin in one liter of boiling water then adding the remaining components and stirring for an additional 10 minutes.

### Plug Test 1

Plug Test 1 was based on methodologies described in U.S. Pat. Nos. 4,060,494 and 4,058,474. Plug Compositions 1 and 2 were transferred to separate glass P-traps. The plugs were allowed to cool for at least 24 hours in the P-traps in order to solidify. After 24 hours, approximately 50 mL of water was added from the entrance side of the P-traps so that the water sat on top each grease plug in the inlet side of the trap. An equal amount (about 2 oz or 56 grams) of the seven cleaners was added to separate P-traps, followed by 8 oz of warm (50° C.) tap water. The experiments were monitored for temperature changes, turbulence, foaming, time to penetration, and rejection of the plugs. In this case, penetration is defined as creating a path measuring a depth of 5-20 mm. Rejection is defined as a complete expulsion of the plug or sufficient fracture of the plug, so that all elements move from the bottom of P-trap to the horizontal arm extending from P-trap. The horizontal arm would represent the discharge waste pipe, which follows the P-trap. Once in this area, the plug is easily flushed out with water.

The results from using the cleaners are summarized in Table 4. During the experiment, the temperature of Cleaner 1 rose during the first two minutes and the first gas bubbles appeared. Within five minutes, Cleaner 1 began to penetrate the grease plug and channeled its way through the grease. Within 15 minutes, the grease plug was fractured and became mobile and capable of being pushed out of the P-trap by the gas pressure being generated or by the addition of further water. Only sulfuric acid based cleaner (Cleaner 3) and a crystalline cleaner formulation (Cleaner 4) containing aluminum and caustic performed as well as the modified Dual Action examples (Cleaners 8, 10, and 11). The advantage to the user is that commercial Cleaners 4 and 5 represent considerable health and safety issues for the consumer while cleaner formulation 1 does not. Liquid caustic and caustic/hypochlorite cleaners (Cleaners 5 and 6) performed considerably slower, requiring hours to penetrate the grease plugs without actually channeling through the grease and displacing the plug.

TABLE 4
RESULTS FROM PLUG TEST 1

| Cleaner | Chemical Type | Results |
|---|---|---|
| Cleaner 1 | caustic/aluminum, | Temperature of drain mixture: 88° C. |
| | peroxygen, | Time to ejection of plug: 5 min |
| | bacteria/carbohydrate | Turbulence |
| | | 400 mL foam |
| | | Plug breaks up into small fragments |
| | | Ejects 5 min |
| Cleaner 2 | caustic/aluminum, | No ejection |
| | peroxygen | |
| Cleaner 3 | sulfuric acid | No ejection |
| | | Plug loosens |
| | | No turbulence |
| | | No foam |
| | | Plug breaks up into small fragments: |
| | | 30 min |
| Cleaner 4 | caustic/aluminum | Temperature of drain mixture: 86° C. |
| | | Time to ejection of plug: 2 hours |
| | | Turbulence |
| | | No foam |
| | | Plug breaks up into small fragments |
| Cleaner 5 | caustic solution | No ejection |
| Cleaner 6 | caustic and | No ejection |
| | hypochlorite | |
| Cleaner 7 | peroxygen | No ejection |
| Cleaner 8 | Cleaner 1 without | Temperature of drain mixture: 30° C. |
| | caustic/aluminum | Time to ejection of plug: >60 min |
| | | Turbulence none |
| | | Foam low |
| | | Plug remains intact |
| | | Ejects 5 min |
| Cleaner 9 | Cleaner 1 plus 10% | Temperature of drain mixture: 90° C. |
| | by weight STPP | Time to ejection of plug: 3 min |
| | | Turbulence |
| | | 450 mL foam |
| | | Plug breaks up into small fragments |
| | | Ejects 3 min |

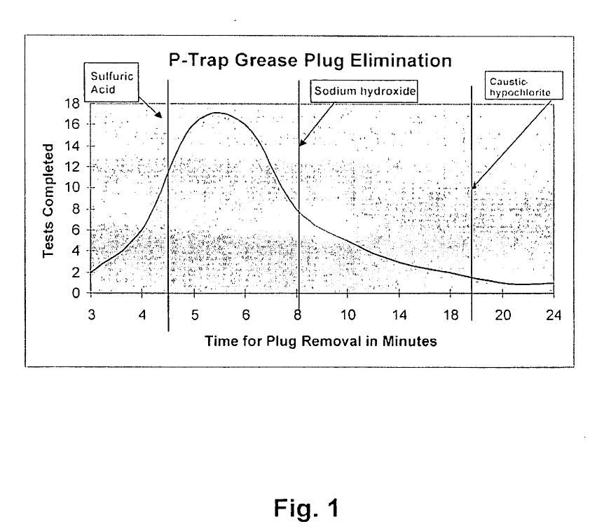

FIG. 1 shows the cumulative result of 60 P-trap plug tests using Cleaner 1 and Plug Composition 1. The 60 tests were performed over an extended period of time by laboratories at NJIT Department of Material Sciences, Hercules Chemical Co., Inc./Passaic, N.J. and Organica Acta/Norristown, Pa., The x-axis represents the time the Cleaner 1 took to remove the test plug. The y-axis represents number of tests conducted. Three vertical lines in the graph represent the average time for plug removal using other commercially available drain cleaners for reference.

FIG. 1 demonstrates that a drain cleaner composition in accordance with in embodiment of the invention is competitive against highly caustic or dangerous acids. As explained above, such a drain cleaner composition also advantageously provides a safe and more environmentally-friendly product.

### Plug Test 2

Plug Test 2 also utilized P-traps, but used only Plug Composition 2. Before the cleaners were added to the P-traps, the P-traps were dissembled and a paraffin sheet was placed between each P-trap and a vertical pipe. The P-traps were then resealed. The paraffin paper prevented any water from entering the P-trap, retaining the water in the vertical tube. A warm sample of Plug Composition 2 was then slowly added to the water in the P-trap and the plug immediately solidified to produce a solid, 1-inch thick plug in the vertical pipe on the surface of the water. 200 mL of water was added to the vertical pipe to rest on top of the grease plug. 56 grams of each cleaner was then added. The results are summarized in Table 5. Only Cleaners 1-6 were tested in this experiment.

TABLE 5
RESULTS FROM TEST 2

| Cleaners | Chemical Type | Results |
|---|---|---|
| Cleaner 1 | caustic/aluminum, | Temperature of drain mixture: 88° C. |
| | peroxygen, | Time to ejection of plug: 5 min |
| | bacteria/carbohydrate | Turbulence |
| | | 400 mL foam |
| | | Plug breaks up into small fragments |
| | | Ejects 3 min |
| Cleaner 2 | caustic/aluminum, | No ejection |
| | peroxygen | |
| Cleaner 3 | sulfuric acid | No ejection |
| | | Plug loosens |
| | | No turbulence |
| | | No foam |
| | | Plug breaks up into small fragments - |
| | | 5 min |
| Cleaner 4 | caustic/aluminum | Temperature of drain mixture: 86° C. |
| | | Time to ejection of plug: 2 hours |
| | | Turbulence |
| | | No foam |
| | | Plug breaks up into small fragments - |
| | | 10 min |
| Cleaner 5 | caustic solution | No ejection |
| Cleaner 6 | caustic and | No ejection |
| | hypochlorite | |

In both Plug Test 1 and Plug Test 2, the amount of turbulence generated by each cleaner was also observed and recorded. The amount of turbulence indicates the increased efficacy of the active components in fracturing the grease plug and promoting channeling through the plug. However, the active ingredients needed to be balanced so that turbulence was restrained to prevent the active solution from backing up and exiting the P-trap into the adjoining sink. Table 6 summarizes the observation of turbulence for each of the cleaners.

TABLE 6
TURBULENCE OBSERVATION

| Cleaners | Chemical Type | Test 1 | Test 2 |
|---|---|---|---|
| Cleaner 1 | caustic/aluminum, | Active | Active |
| | peroxygen, | | |
| | bacteria/carbohydrate | | |
| Cleaner 2 | caustic/aluminum, | Low | Low |
| | peroxygen | | |
| Cleaner 3 | sulfuric acid | None | None |
| Cleaner 4 | caustic/aluminum | Moderate | Moderate |
| Cleaner 5 | caustic solution | None | None |
| Cleaner 6 | caustic and | None | None |
| | hypochlorite | | |
| Cleaner 7 | peroxygen | Active | Active |
| Cleaner 8 | Cleaner 1 without | Low | Low |
| | caustic/aluminum | | |
| Cleaner 9 | Cleaner 1 plus 10% by | Active | Active |
| | weight STPP | | |

### Example 5

### Standardized Published Drain Tests

The following table summarizes the standardized published drain tests of several commercially available cleaners and a drain cleaner composition in accordance with an embodiment of the invention, Dual Action™.

TABLE 7
COMPARISON OF DRAIN CLEANER COMPOSITION VS. COMMERCIAL
CLEANERS
Standardized Published Drain Tests

| | | Pequa HD | | | | | | |
|---|---|---|---|---|---|---|---|---|
| | | Prof. Hair & | 10 Min | | | | | |
| | | Grease | Hair | Flow | | | | |
| | | Kitchen | ZEP | Easy | Drano | | Roebic | Dual |
| | Target | Glugg | Prof. | Clobber | Kitchen | Oxyclean | K-67 | Action |
| Chemical | | | | | Caustic + | AidOx | Bacteria | AidOx + |
| Basis | | | | | Al | | only | Bac |
| Enzyme | | | | | None | None | Yes | Yes |
| Content | | | | | | | | |
| For | | Yes | Yes | No | Yes | Yes | Yes | YES |
| Consumer | | | | | | | | |
| Use | | | | | | | | |
| Hair and | Yes | No | No | Yes | No | No | No | Yes |
| Grease | | | | | | | | |
| Gas | 700 mL | None | None | None | None | None | None | 0.5 L/5 min |
| Generation | | | | | | | | |
| Drain Plug | 3-5 min | Fails | Fails | Pass | Pass | Fail | Fail | Pass |
| Kitchen | | | | | | | | |
| Drain Plug | 5-10 min | Fails | Fails | Pass | Pass | Fail | Fail | Pass |
| Bath | | | | | | | | |
| Turbulence | Foam | None | None | None | None | None | None | Yes |
| | hgt | | | | | | | |
| Heat | >250 cal | <250 | <250 | 400 | 400 | <250 | <250 | 600 |
| Production | | | | | | | | |
| Active O₂ | | None | None | None | None | 50% | None | 35% |
| Content | | | | | | | | |
| Physical | | Liquid | Liquid | Liquid | Solid | Solid | Solid | Solid |
| State | | | | | | | | |
| PH | | 12+ | 13+ | 1 | 12+ | 7 | 7 | 10 |

As can be seen from Table 7, a drain cleaner composition in accordance with an embodiment of the invention produced more heat, gas, and turbulence than all the other commercially available cleaners.

### Example 6

### Hair Removal

Removal of hair is a important function of drain cleaners. Complete degradation of hair is a challenge for the majority of drain cleaners. Highly caustic cleaners and strong acids are only moderately effective. Drain cleaners best suited for hair removal are those based on potassium hydroxide/sodium hypochlorite mixtures, as exemplified by Acuity Brands 10 Minute Hair Remover. The hypochlorite content in this type of fluid is typically 8-10% and the pH is a highly caustic 10-11. In contrast, an embodiment of the disclosed drain cleaner composition can degrade and dissolve hair in a much more facile manner.

A hair removal test was conducted using four different examples. Eight inch lengths of hair samples weighing approximately 1.5 grams were each rinsed with water, washed with shampoo, rinsed, and dried to a constant weight. The hair samples were placed in beakers with 100 grams of water at 35° C. A sample of sixty grams (60 g) each of the selected drain cleaners was added to separate beakers. The hair remained immersed in the drain cleaning solutions for 1-12 hours. The hair samples were then washed and dried to a constant weight. Table 8 summarizes observations from this test.

TABLE 8
WEIGHT LOSS OF HAIR POST-TREATMENT

| | | Wgt | Wgt | % |
|---|---|---|---|---|
| Cleaners | Chemical Type | Before (g) | After (g) | Change |
| Cleaner 1 | caustic/aluminum, | 1.5564 | 0.458 | −71 |
| | peroxygen, bacteria/ | | | |
| | carbohydrate | | | |
| Cleaner 3 | sulfuric acid | 1.6066 | 1.163 | −27 |
| Cleaner 6 | caustic and hypochlorite | 1.5593 | 0.553 | −64.5 |

As shown in Table 8, Cleaner 3, comprising sulfuric acid, dissolved only 27% of the hair. Cleaner 1 dissolved hair to a similar degree as Cleaner 6, a commercial hair remover product.

### Example 7

### Bacterial Survival Rate

To demonstrate the effectiveness of a drain cleaner composition in accordance with an embodiment of the invention as a longer-term maintenance cleaner, a test was conducted extracting bacteria from two different areas of a drain plug. The first area selected was material residing in foam at the top of a drain plug and the second area was material lying near the head of the drain plug. The selected samples were washed with water and then cultured in a medium using the techniques described above. For samples extracted from both areas of the drain composition 90% or more of the microorganism had survived to function as a maintenance cleaner.

What is claimed is:

1. A multi-action drain cleaning composition comprising:
a first component capable of producing an exothermic reaction,
a second component capable of producing an oxidation reaction, and
a third component capable of producing an enzymatic reaction.

2. The multi-action drain cleaning composition of claim 1, wherein
said first component comprises at least one chemical component;
said second component comprises at least one chemical component;
said third component comprises at least one biological component.

3. The multi-action drain cleaning composition of claim 2, wherein said first component comprises at least one base and at least one metal.

4. The multi-action drain cleaning composition of claim 3, wherein said at least one base is sodium hydroxide and said at least one metal is aluminum.

5. The multi-action drain cleaning composition of claim 2, wherein said second component comprises at least one peroxygen compound and at least one organic substrate.

6. The multi-action drain cleaning composition of claim 5, wherein
said at least one peroxygen compound is at least one of sodium percarbonate, sodium persulfate, and sodium perborate, and
said at least one organic substrate is a carbohydrate.

7. The multi-action drain cleaning composition of claim 5, wherein said peroxygen compound comprises about 5-80% by weight of said multi-action drain cleaning composition.

8. The multi-action drain cleaning composition of claim 2, wherein said third component comprises at least one bacterium.

9. The multi-action drain cleaning composition of claim 8, wherein said at least one bacterium is selectively cultured to subsist in an environment comprising said first component and said second component.

10. The multi-action drain cleaning composition of claim 9, wherein said environment is at least one of a peroxygen-rich environment, a high-heat environment, and a high-pH environment.

11. The multi-action drain cleaning composition of claim 10, wherein said bacterium is capable of producing a bio-film that is substantially compatible with said environment.

12. The multi-action drain cleaning composition of claim 11, wherein said third component further comprises a carbohydrate and said bacterium is a lignin-degrading bacterium.

13. The multi-action drain cleaning composition of claim 11, wherein said bacterium produces at least one of a lipase, an amylase, a cellulase, and a protease.

14. The multi-action drain cleaning composition of claim 1 further comprising at least one additive.

15. The multi-action drain cleaning composition of claim 14, wherein said at least one additive is at least one of a diluent, a surfactant, a fatty acid, a fatty acid ester, a carbohydrate, a slip agent, a colorant, a foam suppressant, a fragrance, an anti-caking agent, a binding agent, an abrasive agent, a corrosion inhibitor, and a defoamer.

16. A multi-action drain cleaning composition of claim 14, wherein
said first component comprises caustic beads and aluminum granules,
said second component comprises sodium percarbonate,
said third component comprises lignin-degrading bacteria and dextrose,
said at least one additive comprises at least one of sodium carbonate, sodium lauryl sulfate, and sodium aluminum silicate.

17. A composition for use as a component of a multi-action drain cleaner comprising:
a bacterium selectively cultured to subsist in an environment that is at least one of a peroxygen-rich environment, a high-heat environment, and a high-pH environment.

18. The composition of claim 17, wherein said environment is an aqueous environment including at least one of sodium hydroxide and a peroxygen compound.

19. The composition of claim 18, wherein said bacterium produces at least one of a lipase, an amylase, a cellulase, and a protease.

20. The composition of claim 19, wherein said bacterium is a lignin-degrading bacterium.

21. The composition of claim 19, wherein said bacterium is selectively cultured by a gradient plate method wherein said bacterium is exposed to at least one of an increasing concentration of sodium hydroxide and an increasing concentration of a peroxygen compound.

22. A bio-film for reducing or preventing a buildup of organic material in a pipe system comprising:
a matrix, and
bacteria selectively cultured to subsist in an environment that is at least one of a peroxygen-rich environment, a high-heat environment, and a high-pH environment.

23. The bio-film of claim 22, wherein said matrix comprises polymeric compounds excreted by said bacteria and said matrix coats an interior surface of a pipe system.

24. The bio-film of claim 23, wherein said bacteria metabolize organic material moving through said pipe system and prevent a buildup of said organic material within said pipe system.

25. The bio-film of claim 24, wherein said bacteria are introduced to said pipe system with a multi-action drain cleaning composition that effects within said pipe system said environment that is at least one of a peroxygen-rich environment, a high-heat environment, and a high-pH environment.

26. A pipe system modified to reduce or prevent a buildup of organic material comprising:
a pipe system, and
a bio-film comprising a matrix and bacteria selectively cultured to subsist in an environment that is at least one of a peroxygen-rich environment, a high-heat environment, and a high-pH environment.

27. The modified pipe system of claim 26, where said pipe system is a drain system.

28. A method for treating a clog in a pipe system and for reducing or preventing a buildup of organic material in said pipe system comprising the following step:
providing to a pipe system a multi-action drain cleaning composition comprising
a first component capable of producing an exothermic reaction,
a second component capable of producing an oxidation reaction, and
a third component capable of producing an enzymatic reaction, wherein
said first component comprises at least one base and at least one metal,
said second component comprises at least one peroxygen compound and at least one organic substrate, and
said third component comprises at least one bacterium selectively cultured to subsist in an environment that is at least one of a peroxygen-rich environment, a high-heat environment, and a high-pH environment.

29. The method of claim 28, wherein said at least one bacterium is a lignin-degrading bacterium.

30. The method of claim 29, wherein said at least one bacterium is capable of producing a bio-film that is substantially compatible with said environment.

31. A method for selectively culturing a bacterium to subsist in an environment that is at least one of a peroxygen-rich environment, a high-heat environment, and a high-pH environment comprising the following steps:
providing at least one bacterium to be selectively cultured;
exposing said at least on bacterium to increasing concentrations of at least one of sodium percarbonate and sodium carbonate.

32. The method of claim 31, wherein said at least one bacterium is at least one of Bacillus subtilis ATCC 6051, ATCC 14415, and ATCC 35946, Bacillus licheniformis ATCC 6598 and ATCC 1194, and Bacillus polymyxa ATCC 12060.

33. A method for creating a bio-film for reducing or preventing a buildup of organic material in a pipe system comprising the following step:
providing to a pipe system a multi-action drain cleaning composition comprising
a component capable of producing an enzymatic reaction, wherein
said component comprises at least one bacterium selectively cultured to subsist in an environment that is at least one of a peroxygen-rich environment, a high-heat environment, and a high-pH environment.

34. The method of claim 33, wherein said at least one bacterium is capable of metabolizing organic material and producing a matrix comprising polymeric compounds excreted by said at least one bacterium and said matrix coats an interior surface of said pipe system.

35. The method of claim 34, wherein said at least one bacterium is capable of metabolizing organic material moving through said pipe system and preventing a buildup of said organic material within said pipe system.

36. The method of claim 35, wherein said at least one bacterium is introduced to said pipe system with a multi-action drain cleaning composition that effects within said pipe system said environment that is at least one of a peroxygen-rich environment, a high-heat environment, and a high-pH environment.

37. A method for creating a pipe system with a bio-film for reducing or preventing a buildup of organic material in the pipe system comprising the following steps:
providing a pipe system; and
providing to said pipe system a multi-action drain cleaning composition comprising a component capable of producing an enzymatic reaction, wherein said component comprises at least one bacterium selectively cultured to subsist in an environment that is at least one of a peroxygen-rich environment, a high-heat environment, and a high-pH environment.

38. The method of claim 37, wherein said at least one bacterium is capable of metabolizing organic material and producing a matrix comprising polymeric compounds excreted by said at least one bacterium and said matrix coats an interior surface of said pipe system.

39. The method of claim 38, wherein said at least one bacterium is capable of metabolizing organic material moving through said pipe system and preventing a buildup of said organic material within said pipe system.

40. The method of claim 39, wherein said at least one bacterium is introduced to said pipe system with a multi-action drain cleaning composition that effects within said pipe system said environment that is at least one of a peroxygen-rich environment, a high-heat environment, and a high-pH environment.
